(19) United States
(12) Patent Application Publication
(10) Pub. No.: US 2026/0272631 A1
(43) Pub. Date: Sep. 17, 2026

(54) EVACUATOR MIRROR

(71) Applicant: Ajman University, AJman (AE)

(72) Inventors: Vinay Sivaswamy, Ajman (AE); Arief Cahyanto, Ajman (AE)

(73) Assignee: Ajman University, Ajman (AE)

(21) Appl. No.: 19/076,376

(22) Filed: Mar. 11, 2025

(51) Int. Cl. A61C 17/06; A61B 1/00; A61B 1/015; A61B 1/247; A61C 1/12

(52) U.S. Cl. CPC A61C 17/088; A61B 1/00066; A61B 1/00094; A61B 1/015; A61B 1/247; A61C 1/12

(57) ABSTRACT

An apparatus includes a handle. The handle is hollow and has two apertures. A mirror is attached to the handle. A tube can be enclosed within the handle.

### BACKGROUND

Saliva ejection tubes are commonly used in dentistry for saliva ejection and isolation. Commonly, ejection tubes and mouth mirrors are held separately during clinical practice. One persistent issue is that saliva ejection cannot be done close to the area of the preparation since a dental assistant needs to hold the tube close to the rotary handpiece which impedes the clinician's vision and access during their work.

### BRIEF DESCRIPTION OF DRAWINGS

FIG. 1 is an example perspective view of an evacuator mirror device;

FIG. 2 is another example perspective view of the evacuator mirror device.

FIG. 3 is another example perspective view of the evacuator mirror device;

FIG. 4 is another example perspective view of the evacuator mirror device;

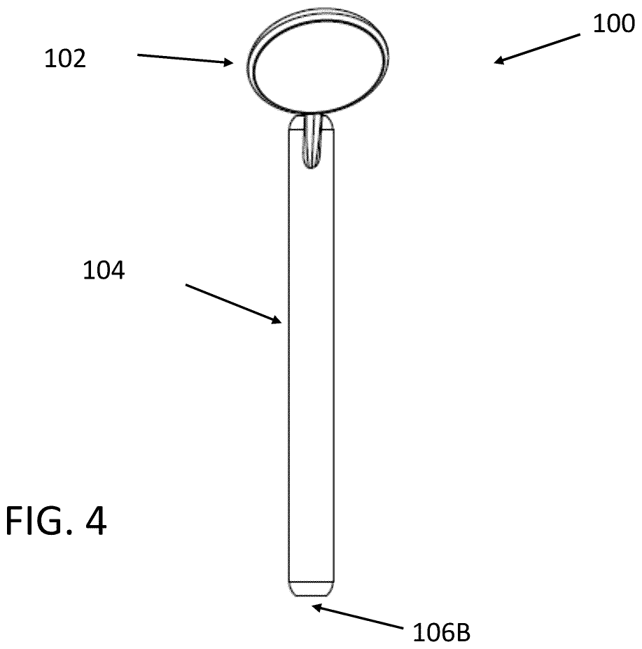

FIG. 5 is another example perspective view of the evacuator mirror device;

FIG. 6 is another example perspective view of the evacuator mirror device; and

FIGS. 7A and 7B are example perspective views of a saliva tube.

### DETAILED DESCRIPTION OF PREFERRED EMBODIMENTS

The following detailed description refers to the accompanying drawings. The same reference numbers in different drawings may identify same or similar elements.

Systems, devices, and/or methods described herein an evacuator mirror device. This innovation combines a conventional mouth mirror handle with a holder for a saliva ejection tube. The handle of the mouth mirror will be made hollow with rounded bevels at the edges to aid in ease of insertion of the saliva ejection tubes. The diameter of the hollow portion will correspond to the diameter of commercially available saliva ejectors to enable seamless adoption in routine clinical practice.

The benefits offered by this innovation include visualization, retraction, isolation and saliva evacuation simultaneously while reducing the need for 2 separate instruments. Other benefits include a reduced need for dental assistant, improved convenience and ease of access for the dental clinician and the advantage of using directly with commercially available saliva ejectors. The mirror portion can also be made with concave mirrors for endodontic purposes.

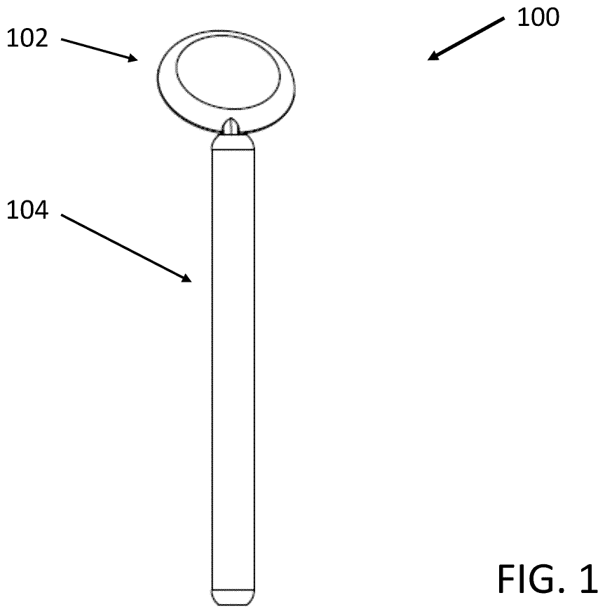

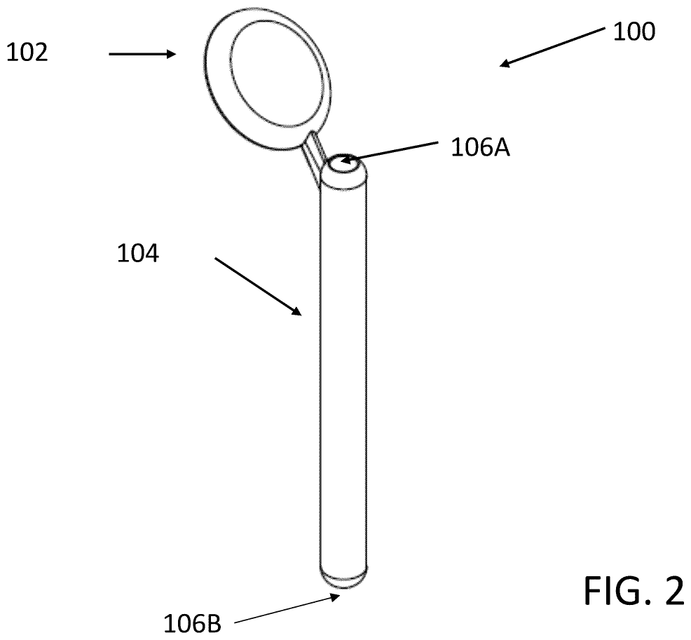

FIG. 1 is an example perspective view of evacuator mirror device 100. As shown in FIG. 1, evacuator mirror device 100 includes mirror 102 and handle 104. FIG. 2 shows another example perspective view of evacuator mirror device 100. As shown in FIG. 2, handle 104 has aperture 106A at the top of handle 104 and aperture 106B. Based on apertures 106A and 106B, handle 104 has a cylindrical space that traverses the length of handle 104 (from the top to the bottom of handle 104) such that handle 104 is hollow such that handle 104 is sufficient to place a tube inside handle 104 and also provide structural strength to handle 104 to be held for use in a dentistry procedure.

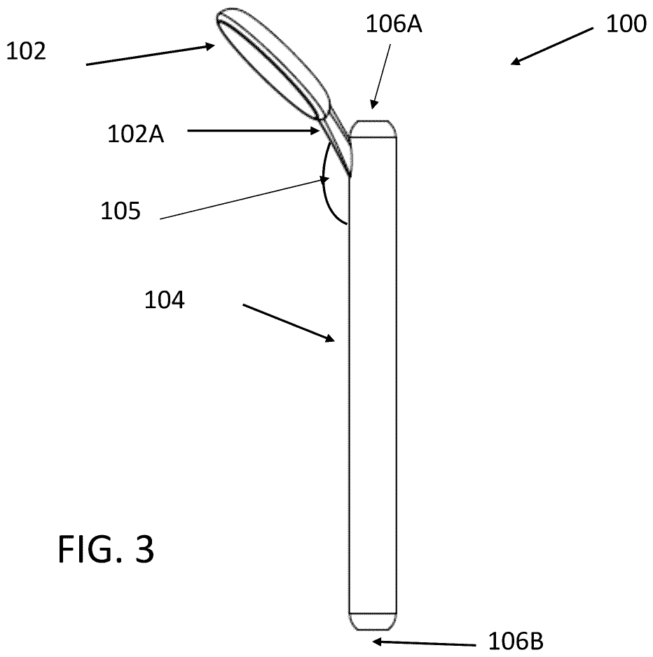

In embodiments, handle 104 has an outer diameter that can range from 6.9 mm (millimeters) to 7.25 mm and an inner diameter that can range from 6.7 mm to 6.8 mm. Handle 104 has a length that ranges from 10 cm to 12.5 cm (centimeters) between apertures 106A and 106B. FIG. 3 shows another example perspective view of evacuator mirror device 100. As shown in FIG. 3, connector 102A is the part of mirror 102 that connects to handle 104. As shown in FIG. 3, connector 102A connects to handle 104 at angle 105. In embodiments, angle 105 may range from 15 to 40 degrees. In embodiments, connector 102A may have a length of 10 mm to 20 mm.

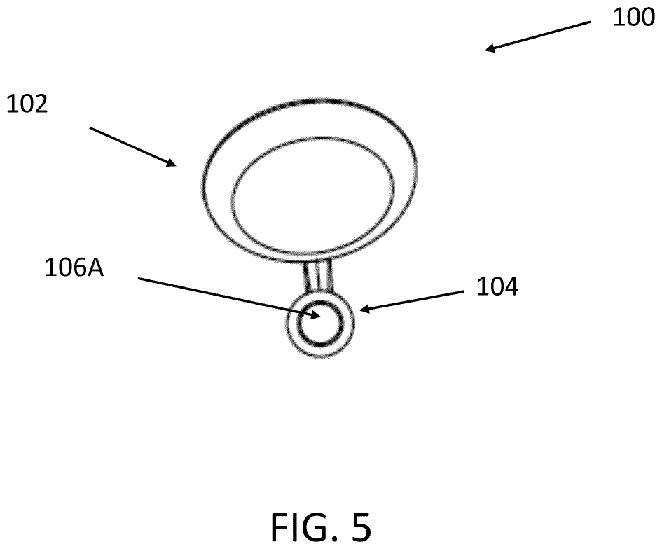

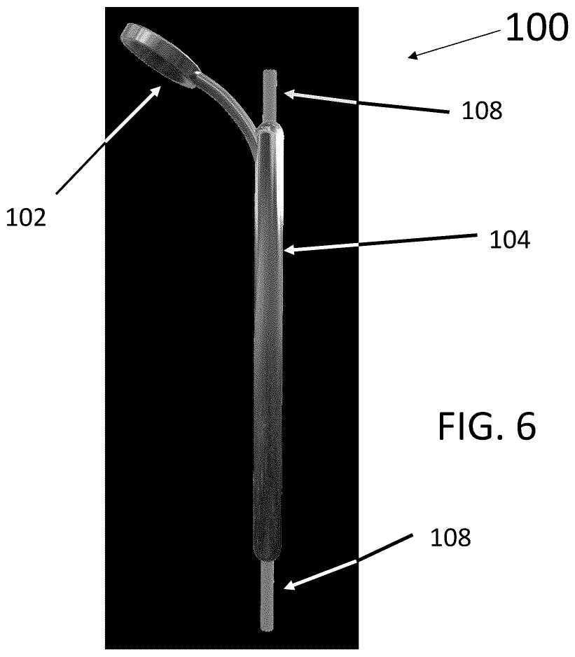

FIG. 5 is another example perspective view of evacuator mirror device 100. As shown in FIG. 5, aperture 106A is shown along with the top view of handle 104. In alternate embodiments, Mirror 102 may be modified to incorporate another mirror on the backend to make a double-sided mirror head. FIG. 6 is another example perspective view of evacuator mirror device 100. As shown in FIG. 6, saliva tube 108 is shown as passing through apertures 106A and 106B; and, thus passing through handle 104.

As shown in FIG. 6, saliva tube 108 is of a greater length than handle 104. In embodiments, saliva tube 108 may be 10 mm to 20 mm above handle 104. Furthermore, in embodiments, saliva tube 108 may be 10 mm to 20 mm below handle 104.

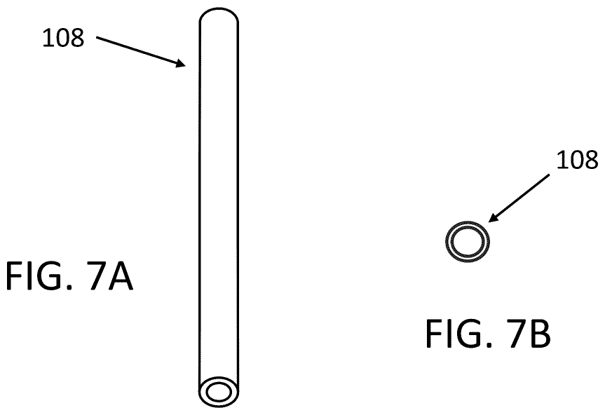

FIGS. 7A and 7B show different view of saliva tube 108. In embodiments, saliva tube 108 may be constructed of plastic (disposable unit) or stainless steel (sterilizable). In embodiments, saliva tube 108 can be connected to a suction device that transfers saliva that passes from a person's mouth through saliva tube 108 and out to the suction device. In embodiments, saliva tube 108 may be rigid in shape being rigid. In alternate embodiments, saliva tube 108 may be flexible in shape.

Even though particular combinations of features are recited in the claims and/or disclosed in the specification, these combinations are not intended to limit the disclosure of the possible implementations. In fact, many of these features may be combined in ways not specifically recited in the claims and/or disclosed in the specification. Although each dependent claim listed below may directly depend on only one other claim, the disclosure of the possible implementations includes each dependent claim in combination with every other claim in the claim set.

No element, act, or instruction used in the present application should be construed as critical or essential unless explicitly described as such. Also, as used herein, the article “a” is intended to include one or more items and may be used interchangeably with “one or more.” Where only one item is intended, the term “one” or similar language is used. Further, the phrase “based on” is intended to mean “based, at least in part, on” unless explicitly stated otherwise.

In the preceding specification, various preferred embodiments have been described with reference to the accompanying drawings. It will, however, be evident that various modifications and changes may be made thereto, and additional embodiments may be implemented, without departing from the broader scope of the invention as set forth in the claims that follow. The specification and drawings are accordingly to be regarded in an illustrative rather than restrictive sense.

What is claimed is:

1. An apparatus, comprising:
a handle, wherein the handle is hollow and has two apertures;
a mirror, wherein the mirror is attached to the handle, wherein the mirror is at a particular angle to the handle, and wherein:
the mirror includes a connector, wherein the connector length ranges from 10 mm to 20 mm,
the connector's other end is attached to the handle, and
the particular angle ranges from 15 to 40 degrees; and
a tube, wherein the tube is enclosed within the handle, wherein:
the tube is flexible in shape,
an exposed top portion of the tube is 10 mm to 20 mm above the handle and an exposed bottom portion of the tube is 10 mm to 20 mm below the handle, and
the tube is attached to an ejector device, wherein saliva is removed from a person's mouth based on attaching the tube to the ejector device and also based on placement of the mirror to determine where saliva is within the person's mouth, wherein the combination of the mirror being attached to the apparatus and the tube being attached to the suction device is configured to:
permit the user of the apparatus to visualize, retract, isolate and removed saliva simultaneously while using only one instrument.

2. The apparatus of claim 1, wherein the tube has a first length and the handle has a second length, wherein the first length is greater than the second length, and wherein:
the handle has an aperture such that the tube is placed inside the handle and the placement of the tube inside the handle provides additional structural strength to the handle,
the handle's outer diameter ranges from 6.9 mm to 7.25 mm and the handle's inner diameter range from 6.7 mm to 6.8 mm, and
the handle's length ranges from 10 cm to 12.5 cm.

3. The apparatus of claim 1, wherein the tube has a first diameter and the handle has a second diameter, wherein the first diameter is smaller than the second diameter.

4. (canceled)

5. A method, comprising:
inserting one end of an apparatus into a person's mouth while the apparatus is held by another person, wherein:
a tube portion of the apparatus is inserted into the person's mouth and the tube portion is enclosed with a handle of the apparatus;
the handle is hollow with apertures that are configured to insert the tube through the handle;
the tube is flexible in shape,
an exposed top portion of the tube is 10 mm to 20 mm above the handle,
and an exposed bottom portion of the tube is 10 mm to 20 mm below the handle;
attaching the other end of the apparatus to a suction device based on the insertion of the tube through the handle;
viewing the inside of the person's mouth, wherein the apparatus includes a mirror, wherein:
the mirror is attached to the handle.
the mirror includes a connector,
the connector length ranges from 10 mm to 20 mm,
the connector's other end is attached to the handle, and
the particular angle ranges from 15 to 40 degrees;
removing saliva from the person's mouth based on the suction device and also based on locating the mirror to determine where saliva is within the person's mouth, wherein the combination of the mirror being attached to the apparatus and the tube being attached to the suction device is configured to:
permit the user of the apparatus to visualize, retract, isolate and removed saliva simultaneously while using only one instrument.
